(19) United States
(12) Patent Application Publication
(10) Pub. No.: US 2022/0091667 A1
(43) Pub. Date: Mar. 24, 2022

(54) IMPERCEPTIBLE MAGNETIC SKIN, MAGNETIC SKIN SYSTEM, AND METHOD OF MAKING MAGNETIC SKIN

(71) Applicant: KING ABDULLAH UNIVERSITY OF SCIENCE AND TECHNOLOGY, Thuwal (SA)

(72) Inventors: Jürgen KOSEL, Thuwal (SA); Abdullah Saud ALMANSOURI, Thuwal (SA); Mohammed Asadullah KHAN, Thuwal (SA); Liam SWANEPOEL, Thuwal (SA)

(21) Appl. No.: 17/420,825

(22) PCT Filed: Jan. 8, 2020
(86) PCT No.: PCT/IB2020/050123

§ 371 Date: Jul. 6, 2021

Related U.S. Application Data
(60) Provisional application No. 62/851,242, filed on May 22, 2019.
(60) Provisional application No. 62/790,096, filed on Jan. 9, 2019.

(51) Int. Cl. G06F 3/01; A61B 5/11; A61B 5/00

(52) U.S. Cl. CPC G06F 3/014; A61B 5/1126; A61B 5/6821; A61B 2562/0223; G06F 3/013; A61B 2562/164; A61B 5/6833

(57) ABSTRACT

A super-flexible and super-stretchable magnetic skin includes a silicone-based elastomeric matrix and a magnetic powder that generates a magnetic field. The magnetic powder is distributed through an entire volume of the silicone-based elastomeric matrix, and the super-flexible and super-stretchable magnetic skin has a Young modulus of less than 1 MPa and a yield strain greater than 200%.

### CROSS-REFERENCE TO RELATED APPLICATIONS

This application claims priority to U.S. Provisional Patent Application No. 62/790,096, filed on Jan. 9, 2019, entitled “MAGNETIC SKIN,” and U.S. Provisional Patent Application No. 62/851,242, filed on May 22, 2019, entitled “MAGNETIC GLOVE,” the disclosures of which are incorporated herein by reference in their entirety.

### BACKGROUND

### Technical Field

Embodiments of the subject matter disclosed herein generally relate to a flexible magnet, and more particularly, to a super-flexible and wearable magnetic skin that easily attaches to the skin or other parts and is used for wireless sensing or touchless interactions.

### Discussion of the Background

The need for wearable electronics has increased significantly in the last two decades. These electronics have a wide range of applications, including tracking the movement and activities of consumers, monitoring the health status of individuals, and serving as a human-to-machine interface. The global market of such devices is expected to reach $160 billion by 2028. However, most commercially existing wearable electronics are in the form of smartwatches and fitness bands, which are bulky and non-flexible.

There are applications (e.g., eye tracking or touchless interaction with a machine) that require an intimate contact between one or more sensors and parts of the body, e.g., the skin. For these applications, the features that would make possible to attach the wearable devices to the skin are biocompatibility, flexibility, light weight, comfort when wearing, and less visibility, in addition to providing accurate measurement and energy-efficient performance. Each wearable device includes electronics that has one or more transducers, which are mainly responsible for the performance, the placement of the device, the nature of the output signal, the complexity of the readout circuit, and the overall power consumption. Thus, while many wearable and flexible sensors have been already developed and are used in the smartwatches and smartbands noted above, there are no directly wearable actuators, i.e., actuators that can be located directly on a part of the human body (e.g., skin), and not on a rigid platform that is mechanically attached to the body.

In this regard, a flexible magneto-electronic device that can be directly attached to the skin is desirable. Flexible magneto-electronics are part of a rapidly progressing field of research, which has brought forward different types of flexible magnets, sensors (such as flexible magnetic tunnel junctions, flexible magnetoimpedance sensors, and flexible hall sensors), and magnetic skins. [1, 2] For example, mixing polydimethylsiloxane (PDMS, i.e., Sylgard 184) with a magnetic powder is one of the most popular methods to achieve flexible magnets. [3] However, the stiffness of the Sylgard imposes limitations to the comfortable attachment and wearability of such flexible magnet. [1]

Thus, there is a need for a new method for making a flexible magnet and a new flexible magnet that can offer extreme flexibility and stretchability, is lightweight, and maintains a high remanent magnetization.

### BRIEF SUMMARY OF THE INVENTION

According to an embodiment, there is a super-flexible and super-stretchable magnetic skin that includes a silicone-based elastomeric matrix and a magnetic powder that generates a magnetic field. The magnetic powder is distributed through an entire volume of the silicone-based elastomeric matrix, and the super-flexible and super-stretchable magnetic skin has a Young modulus of less than 1 MPa and a yield strain greater than 200%.

According to another embodiment, there is a magnetic tracking system for tracking an eye movement, and the magnetic tracking system includes a magnetic skin configured to generate a magnetic field, a magnetic sensor configured to detect the magnetic field and generate an electrical signal that characterizes the magnetic field, and a frame configured to be worn by a user next to an eye. The magnetic sensor is attached to the frame, next to the magnetic skin, and the magnetic skin is attached to an eyelid of the eye.

According to still another embodiment, there is a touchless control system that includes a key pad having plural magnetic sensors, each magnetic sensor of the plural magnetic sensors being associated with a corresponding key, a glove, a magnetic skin attached to the glove, and a controller connected to the plural magnetic sensors and configured to execute a function associated with the key when the magnetic skin is within a given distance range from the corresponding magnetic sensor.

According to yet another embodiment, there is a catheter that includes a body having a tip and a super-flexible and super-stretchable magnetic skin attached to the tip. The super-flexible and super-stretchable magnetic skin includes a silicone-based elastomeric matrix, and a magnetic powder that generates a magnetic field. The magnetic powder is distributed through an entire volume of the silicone-based elastomeric matrix, and the super-flexible and super-stretchable magnetic skin has a Young modulus of less than 1 MPa and a yield strain greater than 200%.

### BRIEF DESCRIPTION OF THE DRAWINGS

For a more complete understanding of the present invention, reference is now made to the following descriptions taken in conjunction with the accompanying drawings, in which:

FIG. 1 illustrates a super-flexible and super-stretchable magnetic skin;

FIG. 2 illustrates the Young modulus and the remanent magnetization of the super-flexible and super-stretchable magnetic skin;

FIG. 3 illustrates a cross-section of the super-flexible and super-stretchable magnetic skin;

FIGS. 4A to 4F illustrate various steps of a process of making the super-flexible and super-stretchable magnetic skin;

FIG. 5 is a flowchart illustrating the process of making the super-flexible and super-stretchable magnetic skin;

FIG. 6 illustrates the stress versus strain for the super-flexible and super-stretchable magnetic skin;

FIG. 7 illustrates the magnetization of the super-flexible and super-stretchable magnetic skin;

FIGS. 8A and 8B illustrate the magnetic flux density versus distance and strain for the super-flexible and super-stretchable magnetic skin and FIG. 8C illustrates the constant magnetic flux density over a number of cycles;

FIGS. 9A to 9E illustrate the behavior of living cells in the presence of the super-flexible and super-stretchable magnetic skin and a reference material;

FIGS. 10A to 10C illustrate a magnetic tracking system for tracking a movement of an eye;

FIG. 11 illustrates magnetic fields recorded with the magnetic tracking system due to the movement of the eye;

FIG. 12A to 12C illustrate various shape and sizes of the super-flexible and super-stretchable magnetic skin;

FIG. 13 illustrates a glove having a super-flexible and super-stretchable magnetic skin;

FIG. 14 illustrates a virtual control key that interacts in a touchless manner with the super-flexible and super-stretchable magnetic skin;

FIG. 15 illustrates a cross-section of the virtual control key; and

FIG. 16 illustrates a medical catheter having the super-flexible and super-stretchable magnetic skin.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a magnetic skin that is made of a magnetic powder and a silicone-based elastomeric matrix (e.g., Ecoflex™ 00-50 silicone from Smooth-On, USA; other silicone-based products from this company may be used). However, the embodiments to be discussed next are not limited to such a silicone-based elastomeric matrix, but other elastomeric matrices may be used as long as the flexibility and stretchability of the final product is compatible with the human skin or other body parts.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a biocompatible magnetic skin is introduced. It offers super-flexibility, super-stretchability, and is lightweight, while maintaining a high remanent magnetization. The flexible magnetic skin is comfortable to wear, can be realized in any desired shape or color, and adds tunable permanent magnetic properties to the surface to which is applied to. The flexible magnetic skin provides remote control functions when combined with magnetic sensors. In one application, the flexible magnetic skin is used to implement a complete wearable magnetic system. For example, eye tracking is realized by attaching the magnetic skin to the eyelid. One advantage of such flexible magnetic skin is that it does not require any wiring, which makes it an extremely viable solution for soft robotics and human-machine interactions. Wearing the magnetic skin on a finger or integrated into a glove allows for remote gesture control or other applications. This type of application opens the door to new control concepts, relevant for people with disabilities, to sterile environments, or to the consumer industry.

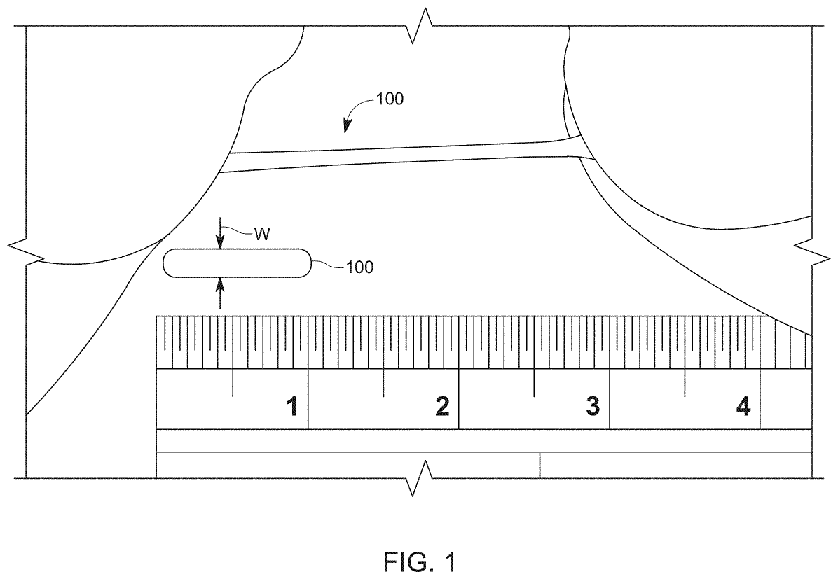

More specifically, a flexible magnetic skin 100 is illustrated in FIG. 1 in two different implementations, one having a length of about 1 cm and the other one having a length of about 3 cm. The flexible magnetic skin 100 may have a width W of about 1 to 5 mm, and a thickness of less than 1 mm. In one embodiment, the thickness of the flexible magnetic skin 100 is less than 0.5 mm. In still another embodiment, the thickness of the flexible magnetic skin 100 is less than 100 micrometers.

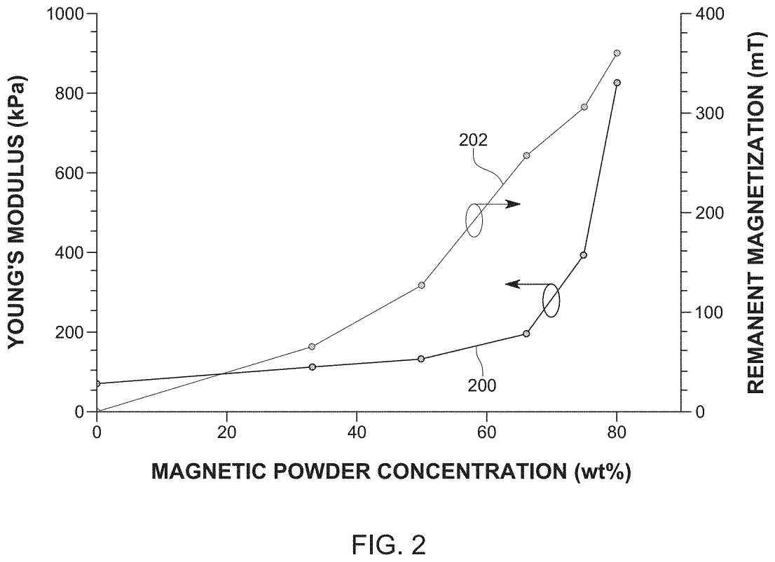

The Young modulus for the flexible magnetic skin 100 is shown in FIG. 2 as curve 200 and the remanent magnetization, measured in milli-Tesla, is shown as curve 202. The flexible magnetic skin 100 is selected to be super-flexible, i.e., the Young modulus is less than 1 MPa, and at the same time, the super-flexible magnetic skin 100 is selected to be super-stretchable, i.e., a yield strain is greater than 200%. In the following, a super-flexible and super-stretchable material is considered to be a material that has the Young modulus less than 1 MPa and the yield strain greater than 200%, respectively. A super-flexible and super-stretchable magnetic skin (also called simply magnetic skin) is defined herein to be a material that includes a magnetic powder distributed throughout a volume of an elastomeric matrix, which has the Young modulus less than 1 MPa and the yield strain greater than 200%.

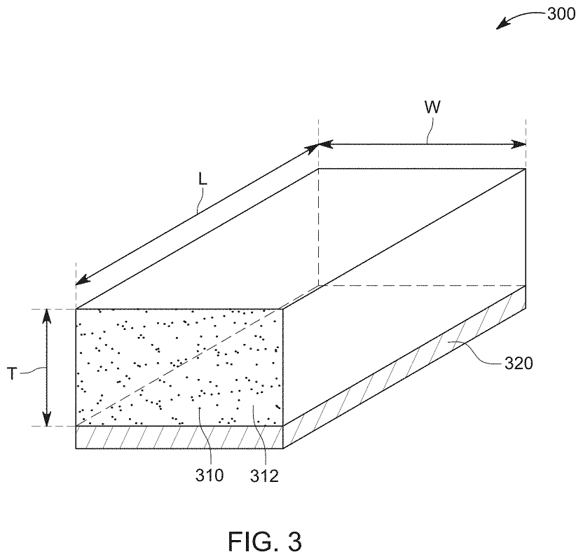

In this regard, FIG. 3 shows a cross-section through a super-flexible and super-stretchable magnetic skin 300 having magnetic particles 310 distributed (substantially uniformly) in a volume of an elastomeric matrix 312. A thickness T of the super-flexible and super-stretchable magnetic skin is less than 1 mm, or less than 0.5 mm, or less than 100 μm, while the length L and the width W can be in the millimeter or centimeter range. The length of the super-flexible and super-stretchable magnetic skin 300 can be even in the meter range. In one application, an adhesive layer 320 may be formed/attached to a side surface of the magnetic skin 300. The adhesive layer 320 may include any known adhesive, e.g., glue, vaseline, etc.

The magnetic particles 310 may include permanent magnetic micro powder NdFeB, wherein the size of each particle is in the micro-meter range. Other compositions may be used for the magnetic particles. The elastomeric matrix 312 may be a silicone-based elastomer, one of the Ecoflex™ silicon rubber, or another material that can exhibit the super-flexibility and super-stretchability discussed above for a thickness less than 1 mm.

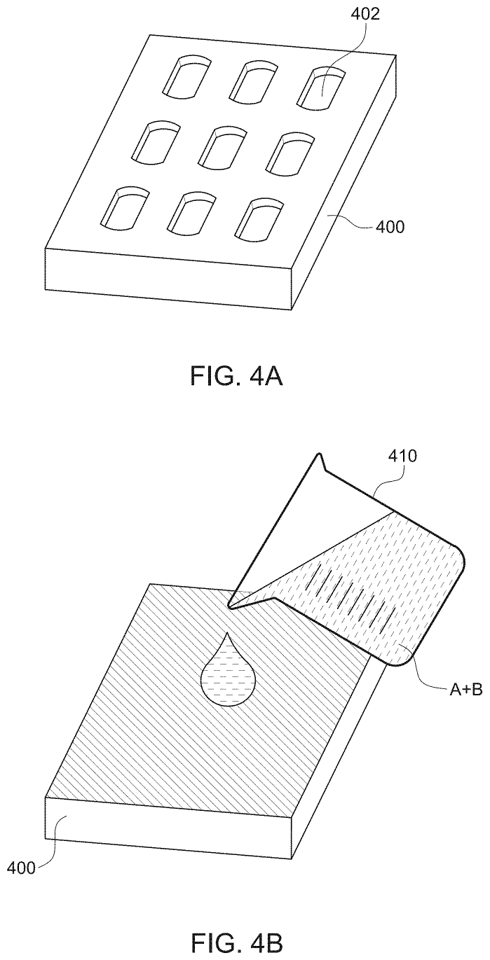

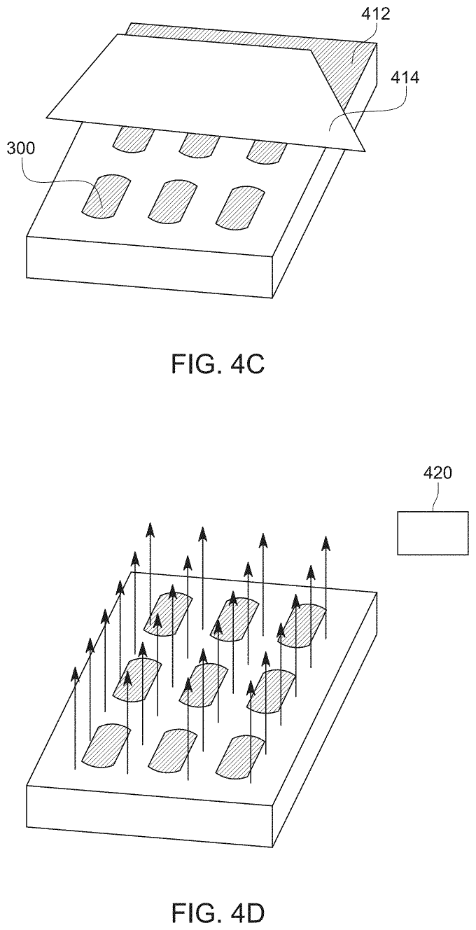

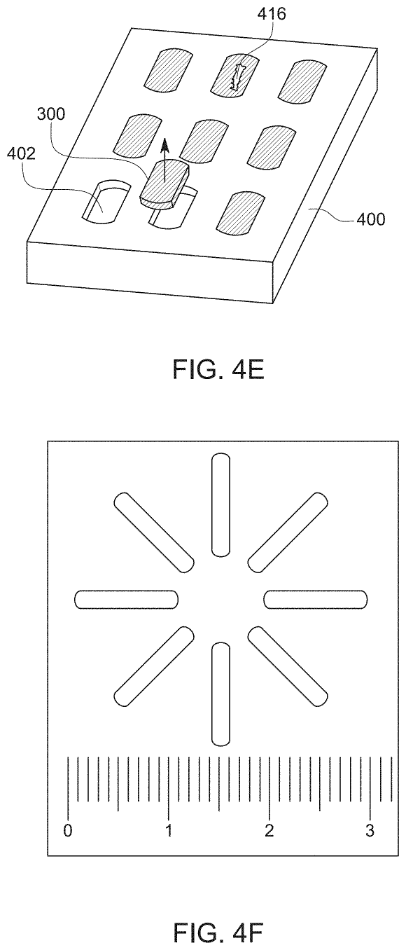

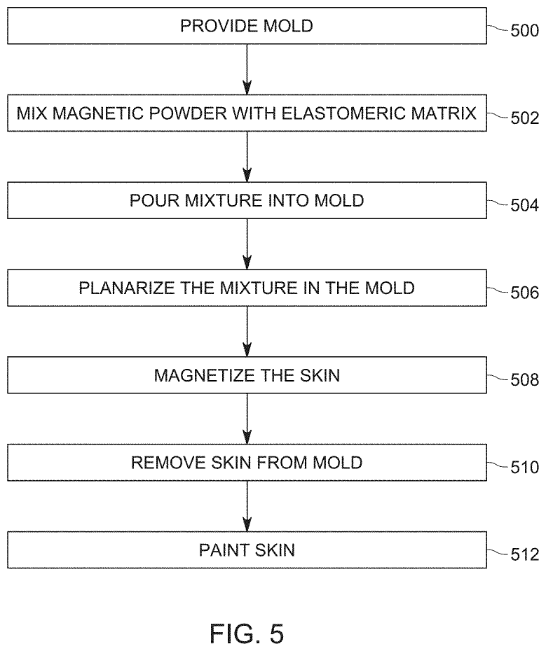

A method for forming the super-flexible and super-stretchable magnetic skin 300 is now discussed with regard to FIGS. 4A to 5. In step 500, a mold 400 with desired shapes 402 and dimensions is provided as illustrated in FIG. 4A. The mold 400 may be 3D printed. In step 502, a quantity A of the magnetic powder is mixed in a vessel 410 with a quantity B of the elastomeric matrix. It is noted that at this time, the elastomeric matrix is in a fluid state. The elastomeric matrix may be obtained by mixing a quantity B12 of a first chemical compound with a quantity B12 of a second chemical compound, according to the recipe for the Ecoflex™ matrix. Mechanical agitation may be used to mix the magnetic powder with the elastomeric matrix. The first and second chemical compounds are in a fluid state and after they are mixed, the mixture slowly becomes a rubber like substance. In one application, the quantity A is equal to the quantity B in terms of mass.

The mixture of the quantity A of the magnetic powder and the quantity B of the elastomeric matrix is then poured in step 504, from the vessel 410 onto the mold 400, to fill the shapes 402, as illustrated in FIG. 4B. To eliminate the possible bubbles in the shapes 402 of the mold 400, it is possible to apply vacuum desiccation for about 15 minutes. In optional step 506, the mixture is planarized and the excess material 412 is removed with a cutter 414 as illustrated in FIG. 4C. The mixture is then cured at room temperature for up to 24 h. The plural super-flexible and super-stretchable magnetic skins 300 are now visible in the shapes 402 of the mold 400. In step 508, the skins 300 are magnetized with an external magnet 420 along a desired direction, as illustrated in FIG. 4D. To obtain a desired magnetization of the skin 300, the external magnet 420 is chosen in one application to generate a magnetic field of about 1.8 T next to the skins.

In step 510 the skins 300 are removed from the mold 400, as shown in FIG. 4E, and they may be painted in step 512, as shown in FIG. 4F, in a desired color. Because of the elastomeric matrix, the magnetic skins 300 may be painted in any desired color. The skins 300 generated in FIG. 4E have a length of about 1 cm, a width of about 2 mm, and a thickness smaller than 1 mm, as illustrated in FIG. 4F.

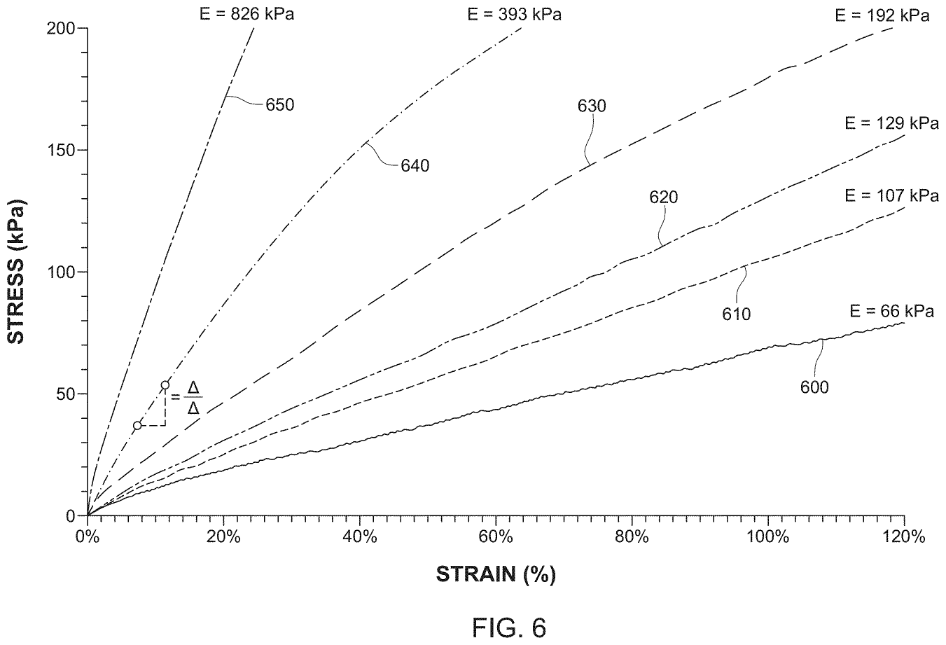

The strain-stress curves for the super-flexible and super-stretchable magnetic skin 300 have been measured and plotted in FIG. 6 for various ratios of the magnetic powder to the elastomeric matrix. The X axis of FIG. 6 plots the strain in percentage while the Y axis plots the stress in kPa. Each composition has its own curve, with curve 600 showing the stress versus strain of the pure elastomer matrix, curve 610 corresponding to 33% by weight of the magnetic powder, curve 620 corresponding to 50% by weight magnetic powder, curve 630 corresponding to 66% by weight magnetic powder, curve 640 corresponding to 75% by weight magnetic powder, and curve 650 corresponding to 80% by weight magnetic powder. Each curve shows its corresponding Young modulus. The small Young modulus indicates the super-flexible behavior of the skin 300. In this respect, it is noted that 1:1 ratio of the magnetic powder to the elastomeric matrix (i.e., curve 620) exhibits a Young modulus 17 times smaller when compared to the PDMS-based flexible magnet with the same concentration of NdFeB. [1] Thus, in one embodiment, the magnetic skin is selected to have a 1:1 ratio of magnetic powder to elastomeric matrix so that the Young modulus is between 90 and 110 kPa.

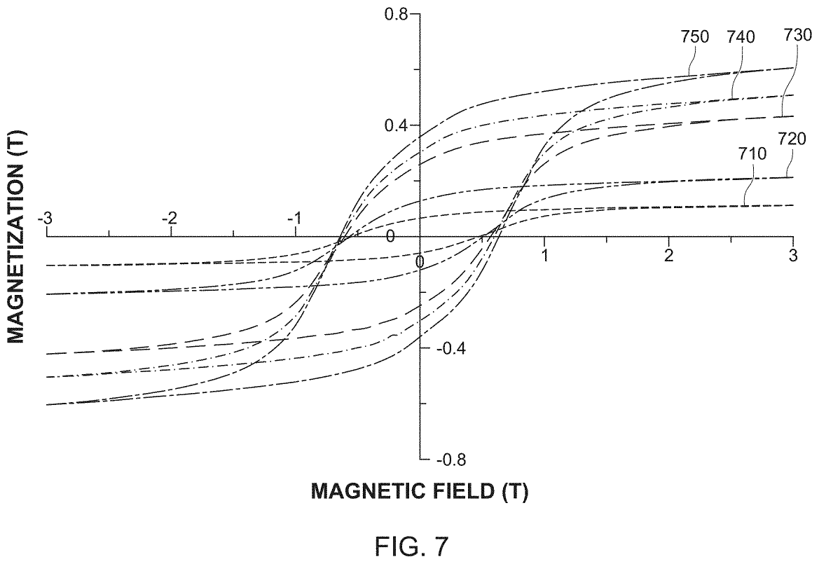

The magnetization curves of the skins 300 considered in FIG. 6 are shown in FIG. 7, with curve 710 corresponding to 33% by weight magnetic particles, curve 720 corresponding to 50% by weight magnetic particles, curve 730 corresponding to 66% by weight magnetic particles, curve 740 corresponding to 75% by weight magnetic particles, and curve 750 corresponding to 80% by weight magnetic particles. It is noted that all curves show the hysteresis shape, which is characteristic for a magnet. The measurement results in FIG. 7 show a maximum remanent magnetization of 360 mT for a 1:4 weight ratio (curve 750).

Based on FIGS. 6 and 7, it is noted that while the 50% mixture is twice as rigid as the native elastomer, the 80% mixture is 12.5 times more rigid than the native elastomer. Thus, the filler concentration has a deleterious effect on the flexibility of the skin, but it also has a beneficial impact on the magnetic properties of the skin. The coercivity of the composite is high (0.56 T, as for pure NdFeB powder) and independent of the filler concentration. This avoids demagnetization of the skin in the presence of magnetic fields that may exist in the sensing environment (such as those in the vicinity of transformers, motors, etc.). The remanent magnetization of the 50% NdFeB skin is approximately one third of the 80% NdFeB skin.

Thereby, going from 50% to 80% NdFeB weight concentration in the skin, increases the remanence by about 200%, while increasing the rigidity by about 540%. Thus, the inventors have concluded that the 1:1 or 50% NdFeB skin offers a good tradeoff between the flexibility and the remanent magnetization, and fits the needs for various applications, e.g., the eye tracking and touchless control, which are discussed later. Moreover, the Young's modulus of the skin with 50% NdFeB is more than 17 times lower than the Sylgard-based PDMS composite magnets [3], which is the most popular polymer matrix used for flexible materials and magnets.

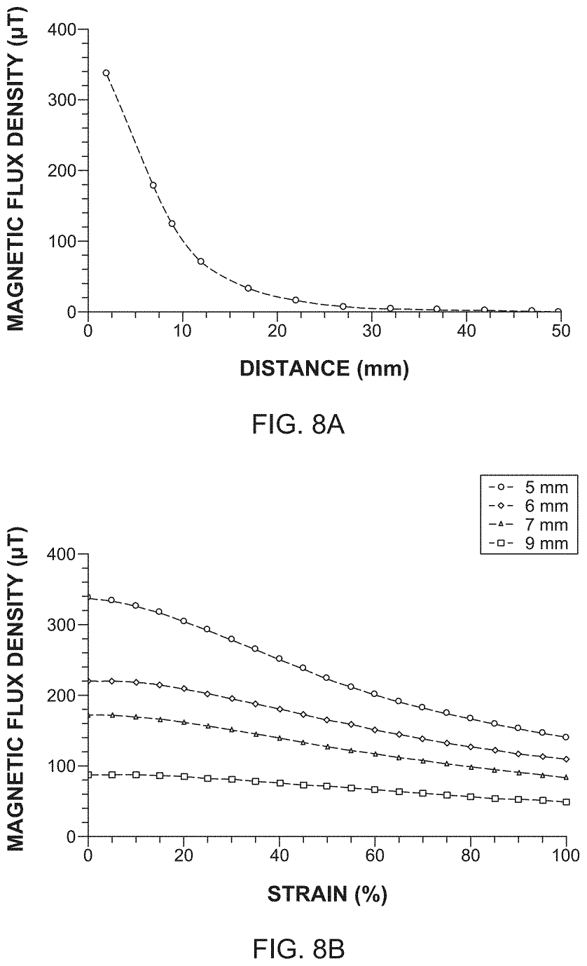

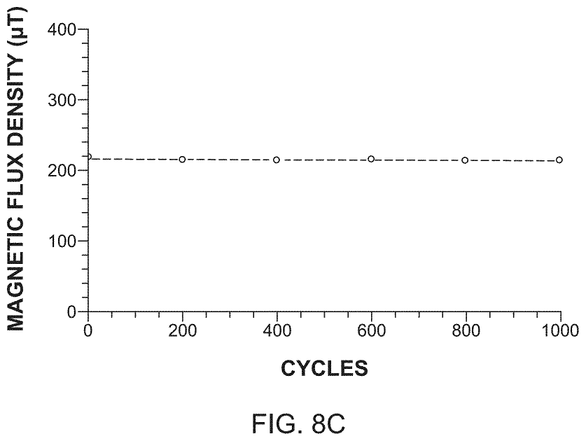

The magnetic properties of the skin 300 were tested over 1,000 stress cycles (i.e., stretching and relaxing) with up to 80% strain. The measurement results presented in FIGS. 8A and 8B illustrate the magnetic flux density dependence on the distance and strain, respectively, and FIG. 8C illustrates a constant magnetic stray field of the magnetic skin 300 over a number of cycles, confirming the mechanical stability of the novel skin. More specifically, the measured stray magnetic field of a 10×2×0.7 mm³ magnetic skin sample, where the magnetization is out of plane (along the 0.7 mm axis), is plotted as shown in FIG. 8A, as a function of a distance between the skin and a magnetic sensor. The measurement results show a reduction in the magnetic field with an increased distance. FIG. 8B shows the magnetic field as a function of the strain for various distances d from the skin. Note, that the strain is along the 10 mm axis in this graph. Stretching the magnetic skin 300 results in less magnetic particles per unit length by thinning the sample, and hence, the magnetic field decreases accordingly.

The magnetic skin 300 made with the method described in FIGS. 4A to 5 can be further processed to become breathable. In this regard, electronic skins (called herein an e-skin) may be worn comfortably and are used for various sensing applications in the healthcare industry. A common consideration with e-skins is the biocompatibility when worn on the skin. Ideally, the e-skin must conform to the topography of the dermal surface and not interfere with the natural physiology of the user's skin. For this reason, the skin must possess breathability, which allows air and moisture from perspiration to move through the e-skin freely. Breathability of the super-flexible magnetic skin 300 is tailored using, for example, one of the following methods:

Cutting: after molding the magnetic skin 300 as illustrated in FIG. 4E, holes 416 (or slots) are cut through the lattice using a laser-cutting tool, such as an ytterbium fiber laser. The ytterbium fiber laser is capable of cutting the super-flexible magnet 300 in any desired shape. Using a laser, it is possible to cut holes into the magnetic skin, hence, enhancing the breathability. The density per square meter and the diameter of the holes 416 can be adjusted to create the required amount of breathability. Note that FIG. 4E shows a single hole 416 formed into the magnetic skin for simplicity.

Punching: after molding the magnetic skin 300, a punching device may be used to induce holes 416 of a specified diameter and density in the magnetic skin.

Molding: the magnetic skin 300 is molded and cured on a surface with high-aspect-ratio needles imbedded into it. After curing, the skin is removed from the mold and the holes 416 are revealed.

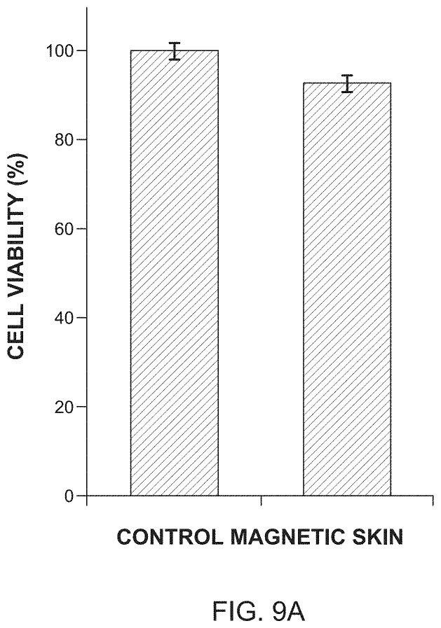

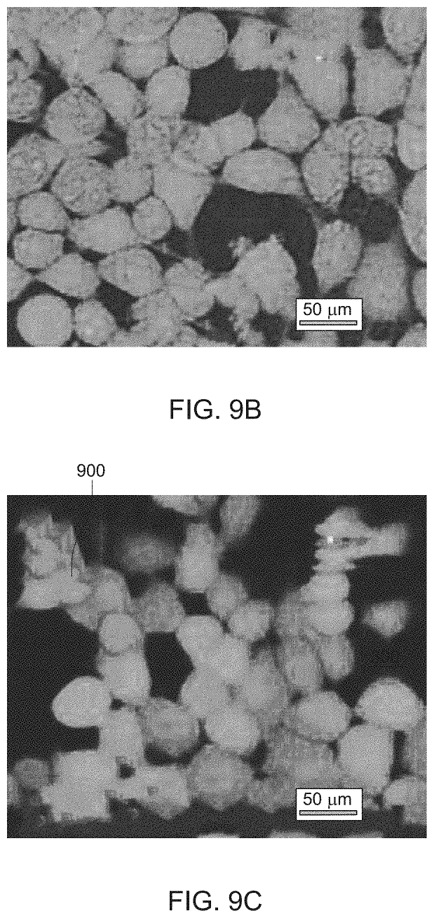

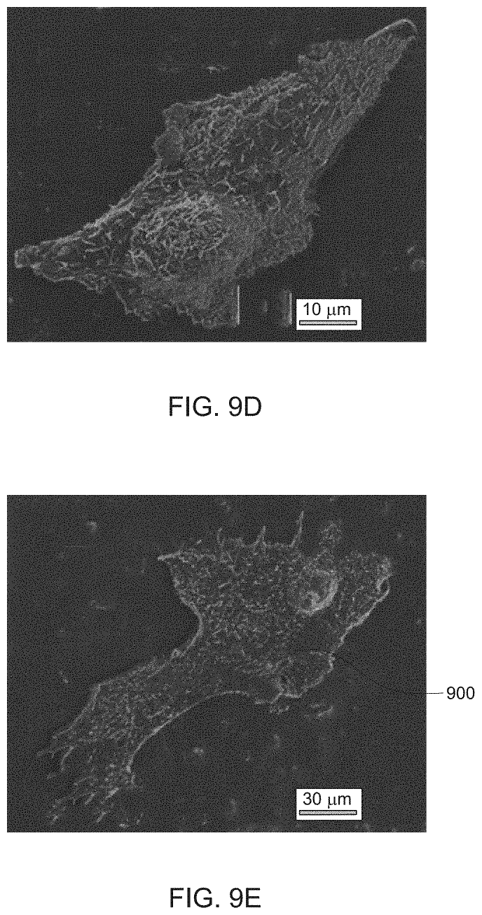

Based on the various features (thickness, weight, magnetic properties, chemical composition, etc.) of the magnetic skin 300 discussed above, it was found to be biocompatible. This feature was assessed using two methods: the PrestoBlue cell viability assay to show quantitative cell viability, and the LIVE/DEAD fluorescence staining method that uses calcein for live cells and ethidium homodimer-1 (EthD-1) for dead cells. The preparation methods of the samples used to determine the biocompatibility followed the practice established in the field. The results of the PrestoBlue assay method, which are plotted in FIG. 9A, show the biocompatibility of the magnetic skin 300 by maintaining a high cell viability (>90%) when cultured for up to 3 days. The error bars in FIG. 9A represent the standard deviation of six replicates. In addition, the fluorescence staining method results, as illustrated in FIGS. 9B and 9C, with FIG. 9B showing a control sample and FIG. 9C showing the cells 900 grown on the magnetic skin 300, show the ability of the HCT 116 cells 900 to grow in a confluent way on the magnetic skin 300. Most of the cells 900 on the magnetic skin are calcein-stained 72 h after growth, indicating a high biocompatibility similar to the control sample shown in FIG. 9B.

Scanning electron microscopy (SEM) imagining is employed to study the morphology of the cells 900 on the magnetic skin 300. In this regard, FIG. 9D show the control sample while FIG. 9E shows the ability of the HCT 116 cells 900 to be elongated on the magnetic skin 300. Furthermore, these cells display a cell membrane rich in both filopodia and lamellipodia, and focal adhesion points similar to the control sample. All these experiments prove that the novel magnetic skin 300 discussed herein is biocompatible and can be safely used with the skin and other body parts of a human being.

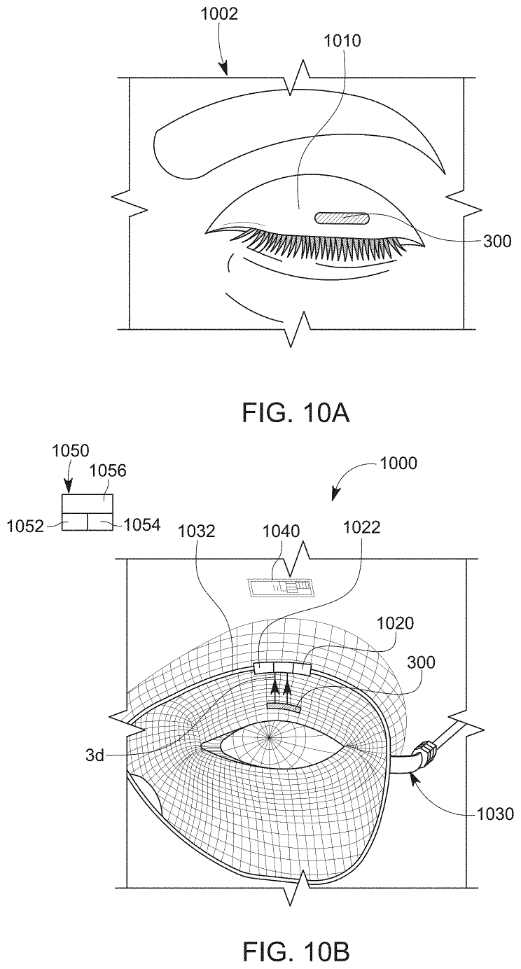

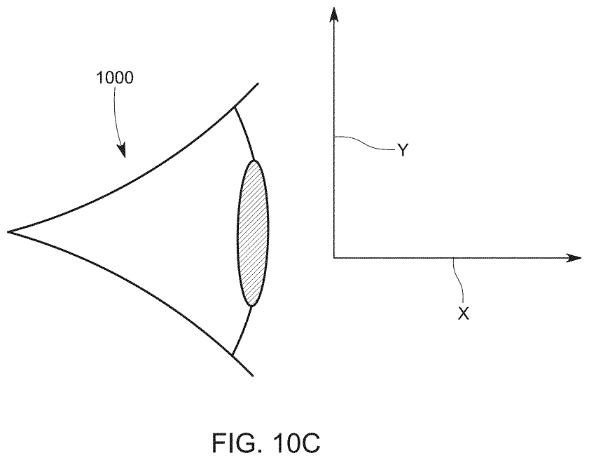

Another application of the magnetic skin 300 is now discussed. Noninvasive and comfortable tracking of blinking eye movements is desirable for various purposes, for example, gaming control, medical investigations, sleep evaluation, marketing, etc. In this regard, a small sample of the magnetic skin 300 was attached to the eyelid 1010 of a human eye 1002, as illustrated in FIG. 10A. In this specific embodiment, the magnetic skin 300 is about 1 cm long, 2 mm wide, and less than 1 mm thick and has a weight of about 19 mg. The magnetic skin is directly attached to the eyelid, for example, with Vaseline. Because of the small size, light weight and super-flexibility and super-stretchability of the magnetic skin, the wearer of the skin did not even notice it. A multi-axis magnetic sensor 1020 is located close by. The magnetic sensor 1020 can be affixed in different convenient locations, such as the frame 1032 of a pair of glasses 1030, or as an electronic tattoo attached to the forehead of the person wearing the magnetic skin 300, or integrated into a sleeping mask for tracking eye movements while sleeping, as shown in FIG. 10B. A magnetic sensor is any device that is capable of measuring a magnetic field and transforming the magnetic field into an electrical signal. In this embodiment, the magnetic skin 300 and the magnetic sensor 1020 form a magnetic tracking system 1000.

In such arrangements, due to the bulge structure of the cornea, any motion of the eye also moves the magnetic skin 300 along a longitudinal axis X and a motion of the eyelid moves the magnetic skin 300 along a parallel axis Y, as shown in FIG. 10C. This movement of the eye, and implicitly the induced movement of the attached magnetic skin changes the magnetic field 301 generated by the magnetic skin 300 and sensed by the multi-axis magnetic sensor 1020. This is so because the eyeball is not perfectly spherical: the cornea introduces a bulged surface. Upon the movement of the eyeball, the cornea pushes the eyelid 1010 and thus, the attached magnetic skin 300 moves outward and inward. As a consequence, the longitudinal magnetic field along the X axis (see FIG. 10C) varies. On the other hand, the magnetic field parallel to the forehead (along Y axis in FIG. 10C) varies only when the user looks upward or downward, even when the eyelid is closed. In an ideal case, this should not change the parallel magnetic field, but moving the eyeball upwards and downwards results in moving the eyelid upwards and downwards too. Therefore, the attached magnetic skin 300 moves and changes the parallel magnetic field on the Y axis. In other words, changes in both the parallel and the longitudinal magnetic fields imply supraversion/infraversion behavior of the eye, while changes in the longitudinal magnetic field only imply levoversion/supraversion.

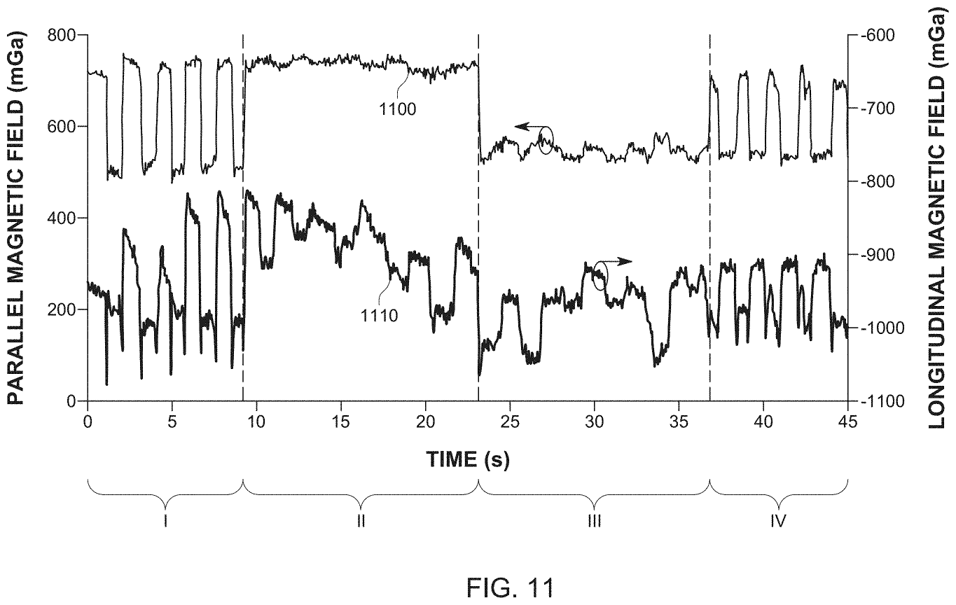

FIG. 11 illustrates the parallel magnetic field 1100 (on the Y axis, on the left side of the figure) and the longitudinal magnetic field (on the Y axis, on the right side of the figure) recorded with the magnetic skin 300 and the multi-axis magnetic sensor 1020, over a period of time of 45 seconds. In the first panel I in FIG. 11, the recorded two magnetic fields correspond to the eyelid being opened and the eye moving up and down. In the second panel II, the recorded two magnetic fields correspond to the eyelid being open and the eye moving right and left. In the third panel III, the recorded two magnetic fields correspond to the eyelid being closed and the eye moving right and left. In the fourth panel IV, the recorded two magnetic fields correspond to the eyelid being closed and the eye moving up and down. It is noted that the two magnetic fields 1100 and 1102 can be used to uniquely determine whether the eyelid is closed or opened, and the eye is moving up or down and right and left. Thus, with the magnetic skin 300 and the multi-axis magnetic sensor 1020 shown in FIG. 10B, it is possible to follow the movement of the eye with minimum intrusion into the life of the person. In one application, the multi-axis magnetic sensor 1020 may have a transmitter 1022 that transmits the collected information to a mobile processing device 1050. The mobile processing device 1050 may be a mobile phone, that has processing capabilities (e.g., a processor 1052 and memory 1054) configured to process the recorded magnetic fields and display the movement of the eye on a display 1056. The mobile processing device may be a server or a computer.

Such an implementation of the magnetic tracking system 1000 has wide applications for a vast range of consumers. For example, eye tracking may be used as a human-computer interface, especially for paralyzed people, in the gaming industry, to analyze individuals' sleep patterns, or to diagnose and wirelessly monitor some eye diseases such as ptosis of the eyelid (i.e., drooping of the eyelid), to observe the behavior of the eye in everyday life, and to monitor driver awareness. As the existing devices are uncomfortable to wear, expensive, invasive, require wired connections or need the eyes to be wide open, the novel magnetic tracking system 1000 would greatly improve any of these applications because of its biocompatibility, lack of wires, and low price.

A survey was conducted to evaluate the comfort level and the impact of having the magnetic skin 300 attached to the eyelid. The survey consisted of 30 volunteers (10 females and 20 males aged from 17 to 36). With a confidence level exceeding 95% (p<0.05, student's t-test), the discomfort level of attaching the magnetic skin 300 onto the eyelid (including the physical and the emotional feelings) is below 1.2, with 0 meaning that the volunteer was not affected by the magnetic skin at all and 5 meaning it had a strong effect. In fact, the small percentage of the participants with discomfort level complained about the adhesive material (Vaseline) that was utilized to attach the magnetic skin to the eyelid, suggesting the use of another less viscous material could remedy this issue. Also, there is no clear difference (i.e., p>0.05) between the comfort level perceived by males and females.

The magnetic skin 300 may be also used to implement a touchless control. In this embodiment, the magnetic skin 300 is attached to a glove, for allowing the user of the glove to control a device by hovering the magnetic skin above a touchless control element. The touchless control element may be a key, switch, pad, etc. This control is achieved without physically touching the control element. This may be especially relevant in laboratories or medical practices, where contamination is of concern. The existing techniques, such as physical buttons, are susceptible to contaminations, and voice-based interfaces usually cannot distinguish between different people speaking in the same room, besides being relatively expensive. Thermal or capacitive techniques are subject to accidental activation, when any part of the anybody is in proximity to the sensor. Body-worn sensors like accelerometers and gyroscopes cannot provide the exact trajectory in addition to the requirement of wearing extra devices. Other proximity sensing techniques usually require computers to analyze the gesture and the position of the hand, which adds to the complexity and the cost of the system, and they are vulnerable to accidental activations.

Although a glove may be used to protect the user from contamination, the problem is that the gloves used by a user in sterile environments are not allowed to be used in a non-sterile environment at the same time. In other words, in sterile environments, the users of the gloves are limited in that their hands cannot touch or make contact with any non-sterile surface. In the laboratory, this may include machine controls or a computer keyboard used to log experimental results. However, a magnetic skin implemented in a glove would address these restrictions of not being able to touch or use any switch or control interface. This is achieved by implementing the magnetic skin as a no-contact alternative. This alternative approach utilizes a thin and lightweight magnet/magnetic strip that is attached to/placed inside a medical/examination glove. The user can use the glove in a sterile environment and interact at the same time with non-sterile systems in a touchless manner through the magnetic skin 300, thus preserving the sterility of the entire glove.

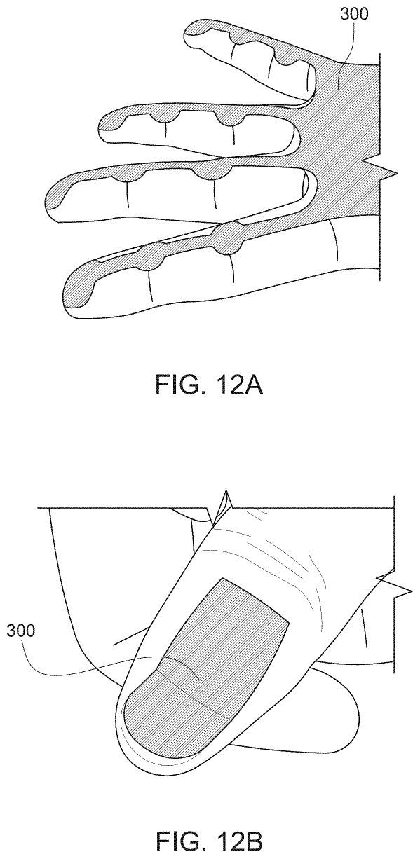

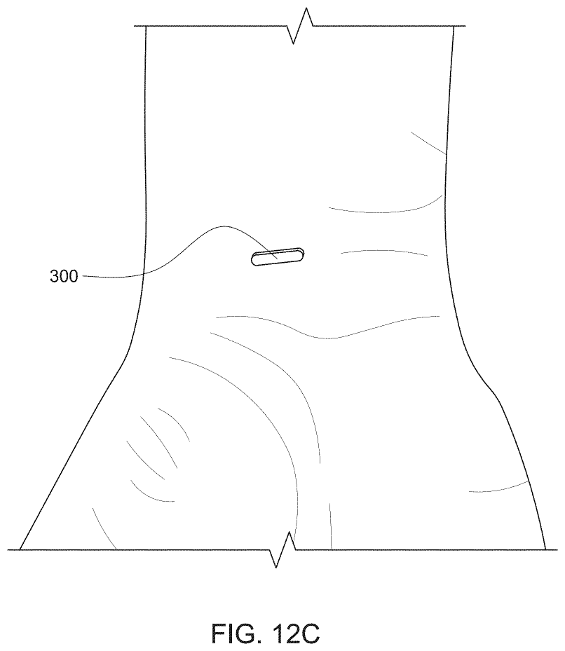

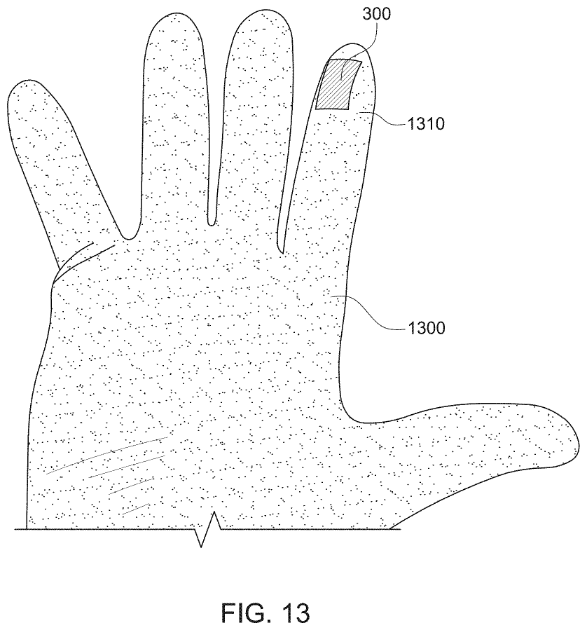

For such applications, the magnetic skin 300 can be utilized for touchless control. It can be comfortably worn directly on any part of the hand, as illustrated in FIGS. 12A to 12B, with the ability to match its color to the skin tone, as illustrated in FIG. 12C. The magnetic skin 300 can be shaped (cut) to any desired shape, depending on the purpose of its application. Also, it is possible to integrate the magnetic skin into a glove 1300 as illustrated in FIG. 13. The size and shape of the magnetic skin 300 may be selected depending on the application. The place on the glove where the magnetic skin 300 is to be attached can also be selected depending on the application. The glove 1300 may be any type of glove as long as the magnetic skin 300 can be attached to it. FIG. 13 shows that the magnetic skin 300 is attached to a tip of a single finger 1310. However, those skilled in the art would understand that the magnetic skin 300 may be attached to any location of the glove, inside or outside. The magnetic skin 300 may be attached with any adhesive to the glove. In one application, the magnetic skin is stitched to the glove. The extreme elasticity of the magnetic skin masks its presence and maintains the original flexibility of the glove. In one embodiment, the entire glove could be made of the magnetic skin 300 material.

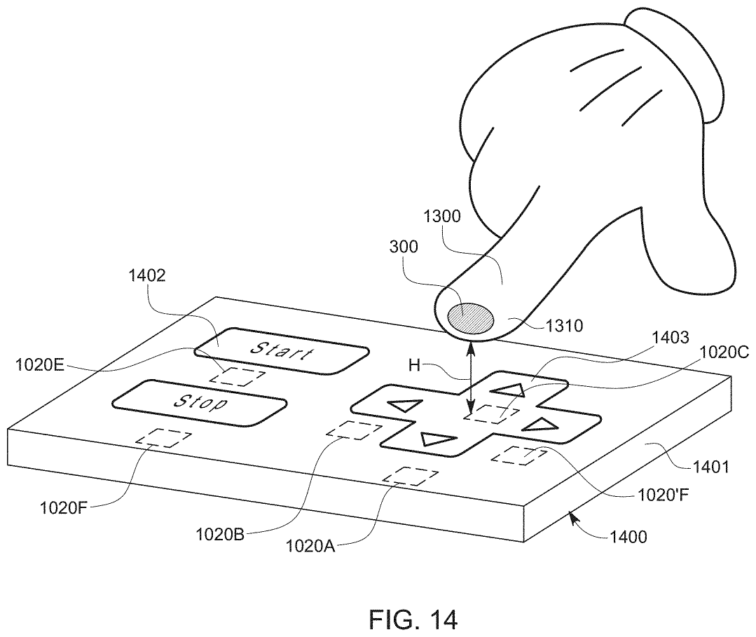

Virtual control keys 1400 were realized using magnetic sensors 1020A to 1020E hidden in a frame 1401, as illustrated in FIG. 14. Each of the magnetic sensors corresponds to a keyboard 1402, which in this embodiment is associated with one of up, down, left, right arrows, start and stop functions. The magnetic skin 300 is attached to a glove 1300, for example, to a single finger 1310. The user of the glove 1300 may place its finger 1310 above the up keyboard 1403, at a distance H. The distance H needs to be larger than zero, but smaller than a given value, that depends on the magnetic field generated by the magnetic skin 300, the sensitivity of the corresponding magnetic sensor 1020C, and also by the type of medium that is present between the magnetic skin 300 and the magnetic sensor. However, irrespective of the specifics of these parameters, there is no need for a direct contact between the magnetic skin 300 and the magnetic sensor 1020C or the frame 1401. For this reason, H>0, which implies a touchless control of the keys, which avoids contamination of the glove from the keyboard.

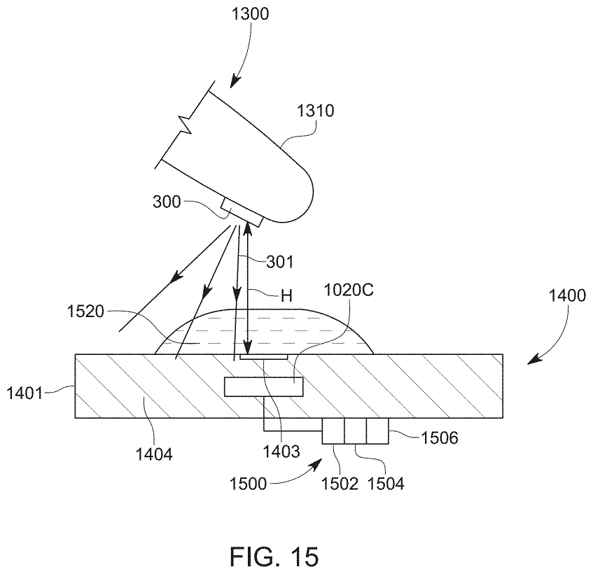

When the magnetic sensor 1020C detects the presence of the magnetic field 301 generated by the magnetic skin 300, as illustrated in FIG. 15, the magnetic field 301 is transformed into an electrical signal by the corresponding magnetic sensor 1020C, and the electrical field is transmitted to a controller 1500 of the virtual control keys 1400. Note that the magnetic sensor 1020C is formed within the material 1404 of the frame 1401. Thus, in this embodiment, the magnetic controllers are hidden from view and not in direct contact with the ambient. The controller 1500 may include a processor 1502, a memory 1504, and a transceiver 1506. The controller 1500 may then performed an action in response to the presence of the magnetic skin 300 in the range defined by H, for example, to move the cursor on a screen in an up direction. It is noted that if the medium 1520 between the virtual control keys 1400 and the magnetic skin 300 is not air, the system still works as long as the magnetic field 301 propagates through the medium 1520. In this regard, it is possible that the entire height H is occupied by the medium 1520. In one application, the entire height H is occupied by the medium 1520 and air. The medium 1520 may include a contaminated liquid, for example, a biological fluid that includes highly dangerous bacteria or viruses. Because the magnetic sensor 1020C is formed within the material 1404 from which the control keys 1400 is formed, there is no danger of contamination for the sensors or the magnetic skin as neither touches the medium. The material 1404 may be any material that allows the magnetic field to propagate through.

Although this dangerous medium 1520 is sitting directly on top of the virtual keys 1400, as illustrated in FIG. 15, the desired key 1403 can be activated when the tip 1310 of the glove 1300 with the magnetic skin 300 is within the pre-determined distance H (threshold distance). This means that this controlling key 1403 cannot be activated unless the magnetic skin 300 is within the threshold distance H. Thus, accidentally pressing or hovering the magnetic skin 300 above the controlling key 1403 with any other part of the body or using other nonmagnetized objects is eliminated.

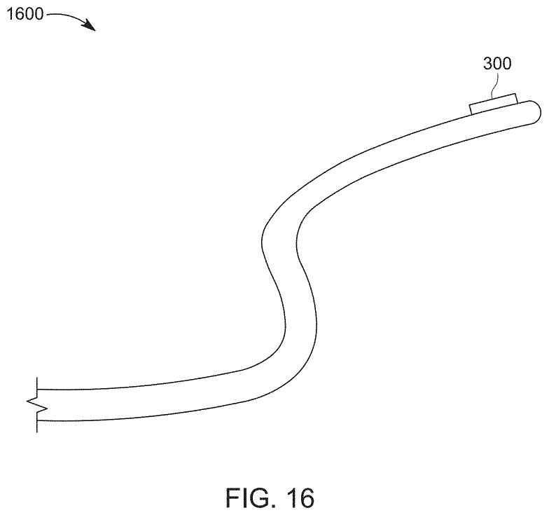

Another application of the magnetic skin 300 discussed above is in the medical field of medical catheters. A catheter is a guiding tube used to deliver medical devices to the targeted location in the human body (i.e., heart). X-ray imaging is currently used to localize the catheter tip inside the human body, but this exposes the patient to large amounts of x-rays combined with contrast agents during the course of the procedure (e.g., surgery). Various alternative approaches are investigated to reduce the use of x-rays, including magnets placed on the tip of the catheter for guiding the catheter using external magnetic field and orientation monitoring. The magnetic skin 300 would be an ideal candidate for such application, given the fact that it is very flexible, lightweight, and thin, whereby all of these parameters can be customized for optimum results. In addition, the magnetic skin 300 is biocompatible and low-cost as well. This means that a catheter 1600 having a body to which the magnetic skin 300 is attached (for example, to its tip), as shown in FIG. 16, would not impede the tip to bend and take the curvature of the vessel in which is deployed, especially at sharp turns. The presence of the magnetic skin on the tip of the catheter would remove the use of the X-ray.

The above embodiments indicate that the imperceptible super-flexible and super-stretchable magnetic skin 300 is biocompatible and highly flexible and stretchable. The viability of cells growing on the magnetic skin remains very high, as evaluated using the PrestoBlue cell viability assay and the LIVE/DEAD fluorescence staining method. It was found that the magnetic skin 300 is up to 17 times more flexible than the more popular Sylgard-based PDMS composites. Combining the features of flexibility, stretchability, and biocompatibility, along with its versatility in shape and color, makes the magnetic skin 300 imperceptible to wear. Thus, it can be comfortably attached to relatively sensitive areas, such as the eyelid. In this case, a nearby multi-axis magnetic sensor can be conveniently integrated into eyeglasses to wirelessly track the movement of the eyeball or the blink of the eye. Furthermore, a touchless control switch may be implemented by attaching the magnetic skin to the fingertip of a glove. This method eliminates accidental activation and contamination of the control keys, while the extreme flexibility of the magnetic skin maintains the elasticity of the glove. The magnetic skin 300 can be combined with flexible and stretchable magnetic sensors on the same substrate, where many different kinds have been realized on polymer substrates before, except for tunnel magnetoresistance sensors, to provide combined remote sensing and actuation.

The disclosed embodiments provide a magnetic skin, magnetic tracking system, and magnetic control system. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### References

[1] G. S. C. Bermudez, D. D. Karnaushenko, D. Karnaushenko, A. Lebanov, L. Bischoff, M. Kaltenbrunner, J. Fassbender, O. G. Schmidt, D. Makarov, Sci. Adv. 2018, 4, eaao2623.
[2] G. S. C. Bermudez, H. Fuchs, L. Bischoff, J. Fassbender, D. Makarov, Nat. Electron. 2018, 1, 589.
[3] A. Kaidarova, M. A. Khan, S. Amara, N. R. Geraldi, M. A. Karimi, A. Shamim, R. P. Wilson, C. M. Duarte, J. Kosel, Adv. Eng. Mater. 2018, 20, 1800229.

1. A super-flexible and super-stretchable magnetic skin comprising:
a silicone-based elastomeric matrix; and
a magnetic powder that generates a magnetic field,
wherein the magnetic powder is distributed through an entire volume of the silicone-based elastomeric matrix, and
wherein the super-flexible and super-stretchable magnetic skin has a Young modulus of less than 1 MPa and a yield strain greater than 200%.

2. The magnetic skin of claim 1, wherein a thickness is less than 1 mm.

3. The magnetic skin of claim 1, wherein the magnetic powder includes NdFeB.

4. The magnetic skin of claim 3, wherein a size of each particle in the magnetic powder is in a micro-meter range

5. The magnetic skin of claim 1, wherein the silicone-based elastomeric matrix includes silicon rubber.

6. A magnetic tracking system for tracking an eye movement, the magnetic tracking system comprising:
a magnetic skin configured to generate a magnetic field;
a magnetic sensor configured to detect the magnetic field and generate an electrical signal that characterizes the magnetic fielder; and
a frame configured to be worn by a user next to an eye,
wherein the magnetic sensor is attached to the frame, next to the magnetic skin, and
wherein the magnetic skin is attached to an eyelid of the eye.

7. The magnetic tracking system of claim 6, further comprising:
a transmitter attached to the frame and electrically connected to the magnetic sensor,
wherein the transmitter is configured to transmit the electrical signal.

8. The magnetic tracking system of claim 7, further comprising:
a controller configured to receive the electrical signal from the transmitter and process the electrical signal to track the movement of the eye.

9. The magnetic tracking system of claim 6, wherein the magnetic skin includes:
a silicone-based elastomeric matrix; and
a magnetic powder that generates a magnetic field,
wherein the magnetic powder is distributed through an entire volume of the silicone-based elastomeric matrix, and
wherein the super-flexible and super-stretchable magnetic skin has a Young modulus of less than 1 MPa and a yield strain greater than 200%.

10. The magnetic tracking system of claim 9, wherein a thickness of the magnetic skin is less than 1 mm.

11. The magnetic tracking system of claim 9, wherein the magnetic powder includes NdFeB.

12. The magnetic tracking system of claim 11, wherein a size of each particle in the magnetic powder is in a micro-meter range.

13. The magnetic tracking system of claim 9, wherein the silicone-based elastomeric matrix includes silicon rubber.

14. The magnetic tracking system of claim 6, wherein the magnetic skin is attached with an adhesive to an eyelid of the eye.

15. A touchless control system comprising:
a key pad having plural magnetic sensors, each magnetic sensor of the plural magnetic sensors being associated with a corresponding key;
a glove;
a magnetic skin attached to the glove; and
a controller connected to the plural magnetic sensors and configured to execute a function associated with the key when the magnetic skin is within a given distance range from the corresponding magnetic sensor.

16. The touchless control system of claim 15, wherein the distance range is different from zero.

17. The touchless control system of claim 15, wherein the magnetic skin generates a magnetic field and the corresponding magnetic sensor measures the magnetic field and transforms the magnetic field into an electrical signal that is transmitted to the controller.

18. The touchless control system of claim 15, wherein the magnetic skin comprises:
a silicone-based elastomeric matrix; and
a magnetic powder that generates a magnetic field,
wherein the magnetic powder is distributed through an entire volume of the silicone-based elastomeric matrix, and
wherein the super-flexible and super-stretchable magnetic skin has a Young modulus of less than 1 MPa and a yield strain greater than 200%.

19. The touchless control system of claim 18, wherein a thickness of the magnetic skin is less than 1 mm, and the magnetic powder includes NdFeB, and a size of each particle in the magnetic powder is in a micro-meter range, and the silicone-based elastomeric matrix includes silicon rubber.

20. (canceled)
